(19) United States
(12) Patent Application Publication
(10) Pub. No.: US 2011/0112129 A2
(43) Pub. Date: May 12, 2011

(54) Peripheral Phenolic Opioid Antagonist

(75) Inventors: Thomas Jenkins, San Carlos, CA (US); Jonathan Wray, San Carlos, CA (US)

(73) Assignee: PharmacoFore, Inc., San Carlos, CA (US)

(21) Appl. No.: 12/743,560

(22) PCT Filed: Nov. 26, 2008
(86) PCT No.: PCTUS2008084978

§ 371 Date: Jul. 13, 2010

Related U.S. Application Data
(63) Continuation-in-part of application No. PCTUS08054127, filed on Feb. 15, 2008, now abandoned.
(60) Provisional application No. 60/990,203, filed on Nov. 26, 2007.
(60) Provisional application No. 61/090,148, filed on Aug. 19, 2008.

(51) Int. Cl. A61K 31/485; C07D 491/08; A61P 1/08; A61P 1/10; A61P 17/04

(52) U.S. Cl. 514/279; 546/39

(57) ABSTRACT

N-Methyldiprenorphine, useful as an opioid antagonist.

This application claims the benefit under 35 U.S.C. 119 of U.S. provisional patent application No. 60/990,203 filed on 26 Nov. 2007, International patent application number PCT/US2008/054127 filed on 15 Feb., 2008 and U.S. provisional patent application No. 61/090,148 filed on 19 Aug., 2008, the contents of which are incorporated herein.

The present invention relates to a novel peripheral phenolic opioid antagonist, to pharmaceutical compositions comprising such an antagonist, to a process for making such an antagonist and to the use of such an antagonist, for example for countering the peripheral side effects of opioids in opioid therapy.

Opioids, for example hydrocodone or phenolic opioids, such as morphine, hydromorphone or oxymorphone, are widely used in the treatment of pain. However, there are problems associated with their use.

One problem associated with the use of opioids is that they can cause unwanted effects that are partly or wholly peripherally mediated, such as constipation, cough suppression, dry mouth, heartburn, myocardial depression, nausea, pruritus, urinary retention, vomiting or bloating, by acting on the peripheral nervous system. These side effects can be countered by co-administering a peripheral μ opioid antagonist, such as the peripheral phenolic opioid antagonist, N-methylnaltrexone, which is commercially available under the trade name RELISTOR™. Commonly the peripherally active opioid antagonists contain a bridgehead quaternary ammonium group, where the centrally active opioid antagonists have a bridgehead amino group. The selective action of these antagonists for peripheral opioid receptors arises from their poor ability to cross the blood brain barrier. This inability to cross-the blood brain barrier tends to be associated with a compromised ability to cross the gut wall, which makes it difficult for an orally administered peripheral antagonist to provide an effective, reproducible response on oral dosing to different patients. In Canada and the United States, N-methylnaltrexone is approved for administration by a subcutaneous injection.

It would be desirable to have an alternative for N-methylnaltrexone, preferably one having improved properties.

N-methylnaltrexone can be prepared by methylating naltrexone (WO 2006/127899). It is chemically related to morphine. Morphine, which is an opioid agonist, has a methyl group at the bridgehead nitrogen atom. Naltrexone has a cyclopropylmethyl group instead of a methyl group. Many of the known orphine (morphine-like) opioid antagonists have a cyclopropylmethyl or allyl group at the bridgehead nitrogen atom. Small modifications to the core structure of orphine agonists or antagonists via addition of small pendant moieties (e.g., methyl) typically do not disrupt their respective activities as agonists or antagonists. In contrast, small modifications to orvinol agonists, such as buprenorphine or diprenorphine, can dramatically alter their function in an unpredictable way (John W. Lewis and Stephen M. Husbands, Current Pharmaceutical Design, 2004, 10, 717-732). Buprenorphine is a systemic μ opioid receptor partial agonist, used for the treatment of pain. Diprenorphine is a systemic μ opioid receptor antagonist. It is extremely potent, and is used primarily to reverse the effects of very potent opioid analgesics, such as etorphine and carfentanil, which are used as tranquilizers in veterinary medicine.

The effect on function of methylating the orvinols appears to have been little studied. WO 2008/059224, published after the priority date of the present invention, asserts that N-methylbuprenorphine has analgesic properties (i.e. is an agonist). However, no test data are provided to confirm this.

Surprisingly, it has now been found that the N-methylated derivative of diprenorphine, N-methyldiprenorphine is a μ opioid antagonist having particularly desirable properties.

According to one aspect, the present invention provides N-methyldiprenorphine (N-MDPN).

N-MDPN has been found to possess high binding affinity for the mu (μ) opioid receptor, to function as a potent antagonist of cellular opioid activity and to possess a particularly desirable pharmacokinetic profile of activity. Based upon the results of initial tests on rats, it is believed that N-MDPN, when dosed orally, may demonstrate a more favorable pharmacokinetic profile than NMTX.

It will be appreciated that N-MDPN contains a quaternary ammonium ion, and may therefore exist and be isolated in the form of a salt. This salt may be represented by the formula (I)
(R)ₙAⁿ⁻ (I)
in which R represents 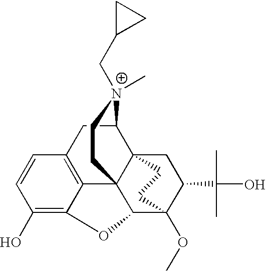
and Aⁿ⁻ represents an anion in which n− represents the charge on the anion.

It will be appreciated that the anion represented by Aⁿ⁻ may be singly charged (for example as in I⁻, Br⁻ or Cl⁻) or multiply charged (for example as in SO₄²⁻ or a polymeric anion, such as an anionic ion exchange resin). Thus, n can be 1, 2, 3, etc, depending upon the charge on the anion. The anion may be derived from an inorganic or organic acid. It will be appreciated that when N-MDPN is to be administered to a patient, the anion should be derived from a pharmaceutically acceptable acid; i.e. the anion represented by Aⁿ⁻ should be a pharmaceutically acceptable anion. However, salts derived from other acids may also be useful as intermediates in the manufacture of a pharmaceutically acceptable salt, for example in a purification step. It will further be appreciated that N-MDPN, being phenolic, may also exist and be isolated in the form of an internal salt. The internal salt and salt forms may together be represented by the general formula (Ia)
(R′)(HₙAⁿ⁻)_{z/n}
in which R′ represents the internal salt form and z represents zero or 1 (corresponding with a salt).

Examples of pharmaceutically acceptable anions include halides (e.g. I⁻, Br⁻ or Cl⁻), sulfate, nitrate, phosphate, carboxylates, sulfonates and phosphonates. Accordingly, N-MDPN may exist and be isolated in the form of a halide (such as an iodide, bromide or chloride), a sulfate, a nitrate, a phosphate, etc, or an internal salt.

It will further be appreciated that N-MDPN may exist and be isolated in the form of a solvate, such as a hydrate. The present invention includes such solvates.

In one embodiment, N-MDPN is in a solid form, for example a crystalline form.

According to another aspect, the present invention provides a process for the preparation of N-MDPN, which comprises methylating diprenorphine.

Diprenorphine may be represented by formula (II)

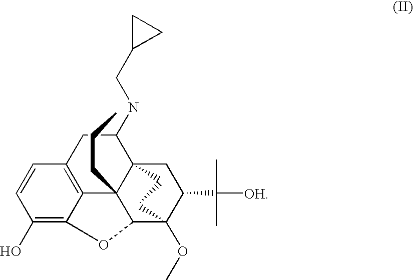

The methylation is conveniently effected by reacting diprenorphine with a methylating agent, for example of formula (III)
CH₃Z (III)
in which Z represents a leaving atom or group, such as a halogen atom, for example an iodine atom, or a hydrocarbylsulfonyloxy group. The reaction is conveniently performed in the presence of an organic solvent, for example a nitrile such as acetonitrile. The temperature is conveniently in the range of from 0° C. to 120° C.

If desired, after the initial formation of N-MDPN, the anion Aⁿ⁻ may be replaced with a different anion, or treated with an appropriate base to afford the internal salt.

It will be appreciated that methylation of diprenorphine affords a quaternary nitrogen atom that is chiral. Accordingly, there are two stereoisomers that can be formed: N-MDPN and its isomer. N-MDPN is the stereoisomer that is the major product of the methylation. N-MDPN can be purified, for example by high performance liquid chromatography. When subject to HPLC purification under the following conditions, as described in Example 1 hereinafter, it is the latter-eluting product, with a retention time of about 2.8 minutes: (RP-18e C18 column (4.6×50 mm); flow rate 1.5 ml/min; mobile phase A: 0.1% trifluoroacetic acid (TFA)/water; mobile phase B 0.1% TFA/CH₃CN; gradient elution from 5% to 100% of B over 9.6 minutes). The absolute configuration of N-MPDN has yet to be determined, but it is believed to be (S). Thus the absolute stereochemistry of the isomer of N-MPDN is believed to be (R).

Accordingly, in another aspect, the present invention provides a method of antagonising the action of an opioid in a patient undergoing opioid treatment, which comprises administering to said patient an effective amount of N-MDPN. In one embodiment, the method is a method of antagonizing the peripheral action of an opioid.

As used herein, the term “patient” includes humans as well as other mammals, such as livestock, zoo animals or companion animals.

In another aspect, the present invention provides N-MDPN for use in therapy.

In yet another aspect, the present invention provides N-MDPN for use as an opioid antagonist. In one embodiment, N-MDPN is for use as a peripheral opioid antagonist.

The amount of N-MDPN administered to the patient will depend upon the species, weight and sex of the patient, the route of administration and the kind and amount of opioid that has been administered.

The opioid may be, for example, hydrocodone, hydromorphone, oxymorphone, morphine, buprenorphine, dihydroetorphine, etorphine or levorphanol. Other examples are nalbuphine, oxycodone, apomorphine, fentanyl, methadone, tramadol, mepiridine, heroin, propoxyphene, alfentanil, sufentanil, remifentanil, butorphanol, pentazocine and nalbuphine.

The opioid may be administered to the patient for any use requiring opioid treatment. For example, an opioid such as hydromorphone can be used, inter alia, to treat or prevent pain including, but not limited to include, acute pain, chronic pain, neuropathic pain, acute traumatic pain, arthritic pain, osteoarthritic pain, rheumatoid arthritic pain, muscular skeletal pain, post-dental surgical pain, dental pain, myofascial pain, cancer pain, visceral pain, diabetic pain, muscular pain, post-herpetic neuralgic pain, chronic pelvic pain, endometriosis pain, pelvic inflammatory pain and child birth related pain. Acute pain includes, but is not limited to, acute traumatic pain or post-surgical pain. Chronic pain includes, but is not limited to, neuropathic pain, arthritic pain, osteoarthritic pain, rheumatoid arthritic pain, muscular skeletal pain, dental pain, myofascial pain, cancer pain, diabetic pain, visceral pain, muscular pain, post-herpetic neuralgic pain, chronic pelvic pain, endometriosis pain, pelvic inflammatory pain and back pain.

N-MDPN may be administered to a patient by any convenient route. In one embodiment, N-MDPN is administered orally or parenterally, for example subcutaneously or intravenously.

The dose at which N-MDPN is administered to a patient will depend upon many factors, including the weight, age, sex and species of the patient, the route of administration and the severity of the condition requiring treatment. It is envisaged that the effective dose will lie in the range of from 0.01 to 25 mg/kg body weight, but appropriate clinical studies will need to be performed before a particular dose can be approved for use by physicians.

N-MDPN may be co-administered with another active ingredient, for example an opioid, such as one of the specific opioids described hereinabove, and/or another analgesic, for example acetaminophen or a non-steriodal anti-inflammatory drug such as ibuprofen.

Generally, N-MDPN will be administered to a patient in a pharmaceutical composition.

In one aspect, the present invention provides a pharmaceutical composition, which comprises N-MDPN and a pharmaceutically acceptable carrier.

Pharmaceutical compositions can take the form of solutions, suspensions, syrups, emulsions, tablets, pills, pellets, capsules, capsules containing liquids, thin films, powders, sustained-release formulations, transdermal patches, suppositories, emulsions, aerosols, sprays, suspensions or any other form suitable for use known to the skilled artisan. In some embodiments, the pharmaceutically acceptable carrier is a capsule (see e.g., Grosswald et al., U.S. Pat. No. 5,698,155). Other examples of suitable pharmaceutical carriers have been described in the art (see Remington's Pharmaceutical Sciences, Philadelphia College of Pharmacy and Science, 19th Edition, 1995). Methods for formulating drugs form part of the common general knowledge of those skilled in the art of pharmacy.

In one embodiment, the pharmaceutical composition further comprises another active ingredient, for example an opioid, such as one of the specific opioids described hereinabove, and/or another analgesic, for example acetaminophen or a non-steriodal anti-inflammatory drug such as ibuprofen.

The ability of N-MDPN to bind to an opioid receptor may be demonstrated using methods known to those skilled in the art, such as those described in Wang, J B, et al., 1994, FEBS Letters 338, 217-222. Using such methods, N-MDPN (prepared as described in Example 1) was found to bind to the μ opioid receptor with an IC₅₀ of 4.2×10⁻⁹ moles per liter (M).

The ability of N-MDPN to antagonize the action of an opioid at the μ opioid receptor may be demonstrated using methods known to those skilled in the art, such as those described in Wang, J B, et al., ibid. In a cellular assay using the human μ opioid receptor expressed on Chinese hamster ovary cells and measuring cyclic adenosine monophosphate (cAMP) by homogeneous time-resolved fluorescence (HTRF® (trademark of Cisbio Bioassays, Inc., Bedford, Mass.)), N-MDPN was found to antagonize the action of 30 nanomolar (nM) DAMGO ([D-Ala2, N-MePhe4, Gly-ol]-enkephalin) with an IC₅₀ of 3.1×10⁻⁷ M.

It will be apparent to those skilled in the art that many modifications, both to materials and methods, may be practiced without departing from the scope of this disclosure. Accordingly, the present embodiments are to be considered as illustrative and not restrictive, and the invention is not to be limited to the details given herein, but may be modified within the scope and equivalents of the allowed claims.

All publications and patents cited herein are incorporated by reference in their entirety.

The following examples illustrate the invention.

### EXAMPLE 1

### Preparation of N-MDPN

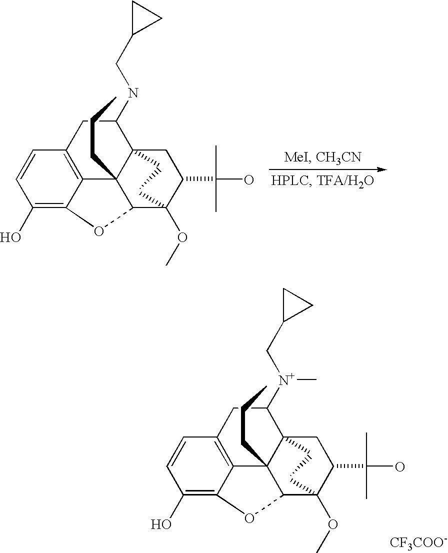

Diprenorphine (available from Johnson Matthey, London, UK) (400 mg, 0.94 mmol) was dissolved in acetonitrile (CH₃CN) (3 ml) at ambient temperature. Methyl iodide (MeI) (0.3 ml, 4.8 mmol) was added. The reaction vial was sealed and heated to 80° C. for 80 hours. Progress of the reaction was monitored by high performance liquid chromatography (HPLC) analysis. This analysis indicated that greater than 50% of the starting material was converted into two stereoisomers (N-MPDN and isomer of N-MPDN), the latter-eluting of which represented about 90% of the converted material and was the desired product, N-MDPN. HPLC analysis (RP-18e C18 column (4.6×50 mm); flow rate 1.5 ml/min; mobile phase A: 0.1% trifluoroacetic acid (TFA)/water; mobile phase B 0.1% TFA/CH₃CN; gradient elution from 5% to 100% of B over 9.6 minutes) provided N-MDPN with a retention time of 2.83 minutes. The remaining reaction mixture was then cooled to ambient temperature and all volatiles were removed in vacuo. The resulting mixture was then submitted to HPLC purification twice using a 50 mm×200 mm C-18 column.

1st Purification from Reaction Mixture:

| | |
|---|---|
| Buffer A: | water with 0.1% TFA |
| Buffer B: | Acetonitrile with 0.1% TFA |
| Flowrate: | 40 mL/min |
| Gradient: | 15-50% B over 70 min. |
| Retention Time: | ˜7.0 min. |

Re-Purification:

| | |
|---|---|
| Buffer A: | water with 0.1% TFA |
| Buffer B: | Acetonitrile with 0.1% TFA |
| Flowrate: | 40 mL/min |
| Gradient: | 10-40% B over 70 min. |
| Retention Time: | ˜18.5 min. Desired fractions were collected and |
| | standard lyophilization afforded N-MDPN in the |
| | form of a trifluoroacetate salt (60 mg, 0.13 mmol, |
| | 14.5% yield, 99.8% HPLC purity). |
| Mass spec: | (m/z) calculated 440.61; observed 440.28. |

1. N-methyldiprenorphine.

2. A process for the preparation of N-methyldiprenorphine, which comprises methylating diprenorphine.

3. A pharmaceutical composition, which comprises N-methyldiprenorphine and a pharmaceutically acceptable carrier.

4. N-methyldiprenorphine, for use in therapy.

5. N-methyldiprenorphine, for use as an opioid antagonist.

6. A method of antagonising the action of an opioid in a patient undergoing opioid treatment, which comprises administering to said patient an effective amount of N-methyldiprenorphine.
